(19) United States
(12) Patent Application Publication
(10) Pub. No.: US 2026/0283959 A1
(43) Pub. Date: Sep. 24, 2026

(54) APPLICATION OF SYNEPHRINE DRY POWDER INHALER IN PREPARATION OF MEDICAMENT FOR TREATING ACUTE LUNG INJURY

(71) Applicant: Anhui University of Chinese Medicine, Hefei (CN)

(72) Inventors: Can PENG, Hefei (CN); Jing ZHANG, Hefei (CN); Chijing ZUO, Hefei (CN); Miaomiao ZI, Hefei (CN); Shen'ao LI, Hefei (CN); Meng SU, Hefei (CN)

(73) Assignee: Anhui University of Chinese Medicine, Hefei (CN)

(21) Appl. No.: 19/554,697

(22) Filed: Mar. 3, 2026

(30) Foreign Application Priority Data
Mar. 20, 2025 (CN) 202510332160.4

(51) Int. Cl. A61K 9/16; A61K 9/00; A61K 31/137; A61K 47/10

(52) U.S. Cl. CPC A61K 9/1682; A61K 9/0075; A61K 31/137; A61K 47/10

(57) ABSTRACT

An application of synephrine dry powder inhaler in the preparation of the medicament for treating acute lung injury is provided. By formulating synephrine into a dry powder inhaler (DPI) dosage form, the disclosure overcomes the problem of low bioavailability of synephrine. After pulmonary inhalation, it directly and actively targets the lungs, increasing the blood drug concentration in the lungs and reducing the distribution of the drug in other tissues, thereby improving drug efficacy and reducing systemic side effects. Simultaneously, the dry powder inhalation method avoids the first-pass effect, which is beneficial to drug stability, and the non-invasive administration route increases patient compliance. The disclosure provides a new approach to the research of pulmonary drug delivery systems and has broad clinical application prospects.

### CROSS REFERENCE TO THE RELATED APPLICATIONS

This application is based upon and claims priority to Chinese Patent Application No. 202510332160.4, filed on Mar. 20, 2025. The entire contents of the application are incorporated herein by reference.

### TECHNICAL FIELD

The present invention belongs to the technical field of pharmaceutical formulation, specifically relating to the application of a synephrine dry powder inhaler in preparation of a medicament for treating acute lung injury.

### BACKGROUND

Acute lung injury (ALI), or its more severe clinical manifestation-acute respiratory distress syndrome (ARDS), is an acute inflammatory lung disease with high annual morbidity and mortality rates. It is a common critical illness with a mortality rate exceeding 40%, seriously threatening patients' lives. ALI is acute respiratory insufficiency caused by damage to alveolar epithelial cells and capillary endothelial cells. ALI is induced by a variety of direct or indirect causes. Direct injuries include pneumonia, aspiration of gastric contents, inhalation of harmful gases, or pulmonary contusion. Indirect injuries are lung injuries caused by other serious diseases such as sepsis, pancreatitis, multiple traumas, or severe systemic infections. Sepsis is the most common cause of acute respiratory distress syndrome in humans. Currently, clinical treatments cannot completely cure ALI; they can only control the progression of the disease and reduce patient suffering and tissue damage. Clinical treatments for ALI primarily include mechanical ventilation, inhalation of vasodilators, and the use of potent anti-inflammatory glucocorticoids.

Synephrine is a natural component of the fruits of plants in the genus Citrus of the Rutaceae family, such as Aurantii Fructus Immaturus, Aurantii Fructus, Citri Reticulatae Pericarpium, and Citri Reticulatae Pericarpium Viride. Among these, it has the highest content in the young fruits of Citrus aurantium L., and is the main active component of extracts from Aurantii Fructus Immaturus, Citrus aurantium L., etc., belonging to ephedrine-type alkaloids. Synephrine shares similarities with endogenous adrenergic receptor agonists adrenaline and noradrenaline in their shared basic structure-phenylethylamine. Due to the structural similarity of synephrine to sympathomimetic amines, synephrine can bind to and activate different α- and β-adrenergic receptors, thereby producing different physiological and pharmacological effects. Pharmacological effects mediated by β-adrenergic receptors can inhibit the expression of pro-inflammatory cytokines in ALI and reduce oxidative stress levels, thus exerting a therapeutic effect.

Synephrine is a white powder with a bitter taste. Its structure contains both a phenolic hydroxyl group and a secondary amino group. It is highly soluble in water or alcoholic solvents, has moderate thermal stability, can react with acids or bases to form salts, and possesses amphoteric properties from a chemical standpoint. It is unstable and easily oxidized under strong acids, strong bases, or high temperatures. After oral administration, pulmonary absorption is low, and bioavailability is limited, requiring higher doses and more frequent dosing, resulting in low patient compliance. Therefore, improving the stability, targeting, and bioavailability of synephrine-based medicaments is an urgent problem to be solved.

### SUMMARY

To address the forementioned shortcomings of the prior art, the present invention aims to provide an application of a synephrine dry powder inhaler in the preparation of a medicament for treating ALI. The present invention, by formulating synephrine into a dry powder inhaler (DPI) dosage form and administering it via pulmonary inhalation, which not only avoids the first-pass effect, thereby enhancing drug stability, but also significantly improves drug targeting and bioavailability.

The present invention is achieved through the following technical solution:

The present invention provides the application of synephrine DPI in the preparation of a medicament for treating ALI.

Preferably, the aerodynamic particle size of the synephrine DPI is 1-5 μm. The atomization performance of a DPI is related to the aerodynamic diameter of the particles. Generally, particles with a diameter less than 0.5 μm are easily expelled with respiration, while particles larger than 10 μm tend to deposit in the upper respiratory tract. Therefore, to achieve good distribution in the deep lung regions, the optimal aerodynamic diameter of the inhaled particles is 1-5 μm.

Preferably, the synephrine DPI has a fine particle fraction (FPF) of 28-33%, an emptying rate of 94-98%, an inhalable fraction of 60-70%, and an effective in vitro lung deposition rate of 18-20%. The synephrine DPI prepared by the present invention possesses suitable physicochemical properties including an emptying rate and FPF, that meet the requirements for pulmonary inhalation administration, and can be effectively deposited in the lungs.

The method for preparing the synephrine DPI according to the present invention includes the following steps:
(1) dissolving a synephrine active pharmaceutical ingredient (API) in a good solvent to form good solvent phase A, and using n-hexane as an antisolvent phase B;
(2) adding the good solvent phase A dropwise to the antisolvent phase B under stirring conditions to obtain a uniform suspension; and
(3) centrifuging the suspension obtained in step (2), and subsequently precipitating and drying to obtain the synephrine DPI.

The present invention, based on the solubility of synephrine API in different polar solvents, employs an antisolvent recrystallization method. This method eliminates the need for other carriers and excipients, enabling synephrine to form a DPI with uniform particle size, good morphology, and stable chemical properties during recrystallization. Compared to traditional preparation methods, the present invention offers a shorter reaction time, simpler operation, and is environmentally friendly. it eliminates the use of materials toxic to humans or the environment and provides a new approach for the industrial-scale production of DPIs using the antisolvent recrystallization method.

In step (1), the good solvent is one or more of water, anhydrous ethanol, acetonitrile, isopropanol, or n-butanol, more preferably anhydrous ethanol.

Preferably, instep (1), the concentration of the good solvent phase A is 10-20 mg/mL.

Preferably, instep (2), the stirring speed is 600-1200 rpm, and the stirring time is 2-10 h.

Preferably, instep (2), the volume ratio of the good solvent phase A to the antisolvent phase B is 1:8-12.

Preferably, instep (3), the drying method is constant temperature drying at 30-40° C.

Compared to the prior art, the present invention has the following advantages:

The present invention provides a new application of synephrine DPI in the preparation of a medicament for treating ALI. By formulating synephrine into a DPI dosage form, the problem of low bioavailability of synephrine is overcome. After pulmonary inhalation, it directly and actively targets the lungs, increasing the blood drug concentration in the lungs and reducing the distribution of the drug in other tissues, thereby improving drug efficacy and reducing systemic side effects. Simultaneously, the dry powder inhalation method avoids the first-pass effect, which is beneficial for drug stability, and the non-invasive administration route increases patient compliance. The present invention provides a new approach for research on pulmonary drug delivery systems and has broad clinical application prospects.

### BRIEF DESCRIPTION OF THE DRAWINGS

FIG. 1 shows the effect of stirring speed on the particle size of the synephrine DPI;

FIG. 2 shows the effect of stirring time on the particle size of the synephrine DPI;

FIG. 3 is a scanning electron microscope image of the synephrine DPI;

FIG. 4 is the particle size distribution diagram of the synephrine DPI;

FIG. 5 shows the powder X-Ray diffraction (PXRD) patterns of synephrine API and DPI;

FIG. 6 shows the differential scanning calorimetry (DSC) curves of synephrine API and DPI;

FIG. 7 shows the Fourier transform infrared spectroscopy (FTIR) spectra of synephrine API and DPI;

FIG. 8 shows the moisture adsorption curves of synephrine API and DPI;

FIG. 9 shows the in vitro deposition distribution diagram of synephrine API and DPI;

FIG. 10 shows the effect of the synephrine dry powder inhaler (SYN-DPI) on the survival rate of RAW264.7 cells;

FIGS. 11A-11D show the effects of SYN and SYN-DPI on the expression of pro-inflammatory cytokines in the LPS-induced RAW264.7 inflammation model;

FIG. 12 shows the effects of SYN and SYN-DPI on the wet-to-dry weight ratio of lung tissue in the LPS-induced rat ALI model;

FIG. 13 shows the pathological HE and Masson sections of the lung tissue of the LPS-induced rat ALI model treated with SYN and SYN-DPI;

FIG. 14 shows the effect of SYN-DPI on the total protein content in BALF of ALI rats;

FIGS. 15A-15F show the effects of SYN and SYN-DPI on serum inflammatory cytokines in the LPS-induced rat ALI model;

FIGS. 16A-16C show the effects of SYN and SYN-DPI on oxidative stress levels in the LPS-induced rat ALI model;

FIGS. 17A-17F show the immunohistochemical detection results of NF-κB, Nrf2 and p38 MAPK protein expression in lung tissue of ALI rats in different groups;

FIG. 18 shows the average plasma drug concentration-time curve of synephrine in rats after different administration methods.

### DETAILED DESCRIPTION OF THE EMBODIMENTS

The technical solution of the present invention will be clearly and completely described below with reference to the embodiments of the present invention. The described embodiments are merely some embodiments of the present invention, and not all embodiments. All other embodiments obtained by those skilled in the art based on the embodiments of the present invention without creative effort are within the scope of protection of the present invention.

### Example 1: Preparation of Synephrine DPI

An excess of 1.00 g of synephrine API was dissolved in 50 mL of anhydrous ethanol. After sonication (300 W, 40 kHz) for 15 min, the supernatant was collected by centrifugation to obtain a saturated alcoholic solution of synephrine. Under stirring conditions, the saturated alcoholic solution of synephrine was added dropwise to an n-hexane solution at a volume ratio of 3:30 (mL:mL). The stirring time started from the moment the saturated alcoholic solution of synephrine was completely added to the hexane. After stirring was completed, the mixture was centrifuged, the supernatant was discarded, and the precipitate was dried in a 40° C. forced-air drying oven for 24 h to obtain the synephrine DPI.

### I. Screening of the Preparation Process for Synephrine Dry Powder Inhaler (SYN-DPI)

### 1 Reagents (see Table 1)

TABLE 1
Experimental Materials (Production Lot Manufacturer Number)

| | |
|---|---|
| Synephrine API | Shanghai Macklin Biochemical |
| (C15086459) | Technology Co., Ltd. |
| Synephrine Control | Shanghai Macklin Biochemical |
| (C13531235) | Technology Co., Ltd. |

### 2 Methods

### 2.1 Determination of Synephrine Solubility by High Performance Liquid Chromatography

### 2.1.1 Liquid Chromatographic Conditions

Column: Agilent C18 (250 mm×4.6 mm, 5 μm) column; mobile phase: 0.1% formic acid (A)-acetonitrile (B); detection wavelength: 273 nm; flow rate: 1 mL/min; column temperature: 30° C.; injection volume: 10 μL; isocratic elution: 1 min to 10 min; 97% A.

### 2.1.2 Determination of the Solubility of Synephrine API in Different Solvents

Excess synephrine API was weighed and placed in an EP tube. Then, 1.00 mL of each of the following solutions was added: water, anhydrous ethanol, isopropanol, ethyl acetate, n-butanol, n-octanol, acetonitrile, and n-hexane. The mixture was sonicated (300 W, 40 kHz) until no further dissolution occurred. The tube was then centrifuged at 6000 r min⁻¹ for 15 min, filtered through a 0.22 μm microporous membrane, and the filtrate was diluted with methanol and analyzed under liquid chromatography conditions. The solubility of synephrine in different solvents was calculated.

### 2.2 Investigation of Stirring Speed

First, an excess of 1.00 g of synephrine API was dissolved in 50 mL of anhydrous ethanol. After sonication (300 W, 40 kHz) for 15 min, the supernatant was collected by centrifugation to obtain a saturated alcoholic solution of synephrine. The saturated alcoholic solution of synephrine was added dropwise to an n-hexane solution at a volume ratio of 3:30 (mL:mL). The mixture was stirred at speeds of 600, 900, and 1200 rpm, respectively. After stirring was completed, the mixture was centrifuged, the supernatant was discarded, and the precipitate was dried in a 40° C. forced-air drying oven for 24 h to obtain the synephrine DPI.

### 2.3 Investigation of Stirring Time

First, an excess of 1.00 g of synephrine API was dissolved in 50 mL of anhydrous ethanol. After sonication (300 W, 40 kHz) for 15 min, the supernatant was collected by centrifugation to obtain a saturated alcoholic solution of synephrine. The saturated alcoholic solution of synephrine was added dropwise to an n-hexane solution at a volume ratio of 3:30 (mL:mL). The stirring times were set at 0.5, 1, 1.5, 2, and 6 h, respectively. After stirring was completed, the mixture was centrifuged, the supernatant was discarded, and the precipitate was dried in a 40° C. forced-air drying oven for 24 h to obtain the synephrine DPI.

### 2.4 Investigation of Particle Size

Appropriate amounts of synephrine API and synephrine DPI were taken and their particle size distribution was measured using a laser particle size analyzer via the dry dispersion method.

### 3 Results and Analysis

### 3.1 Solubility of Synephrine API in Different Solvents

The solubility of synephrine API in different solvents is shown in Table 2. The results indicate that synephrine has a high solubility in anhydrous ethanol and water, a low solubility in n-octanol and ethyl acetate, and is almost insoluble in n-hexane. Based on the principle of antisolvent precipitation, that is, good solvents have high solubility and antisolvents have poor solubility. Considering the ease of removing residual solvent after purification, anhydrous ethanol was ultimately chosen as the good solvent and n-hexane as the antisolvent for preparing synephrine DPI.

TABLE 2

| Solvent | Solubility (mg/mL) |
|---|---|
| Water | 13.44 ± 0.02 |
| Anhydrous Ethanol | 17.20 ± 0.27 |
| Acetonitrile | 10.62 ± 0.06 |
| Ethyl Acetate | 0.64 ± 0.01 |
| Isopropanol | 7.06 ± 0.12 |
| n-Butanol | 8.76 ± 0.02 |
| n-Octanol | 3.66 ± 0.01 |
| n-Hexane | — |

### 3.2 Effect of Stirring Speed on Particle Size Distribution of Synephrine DPI

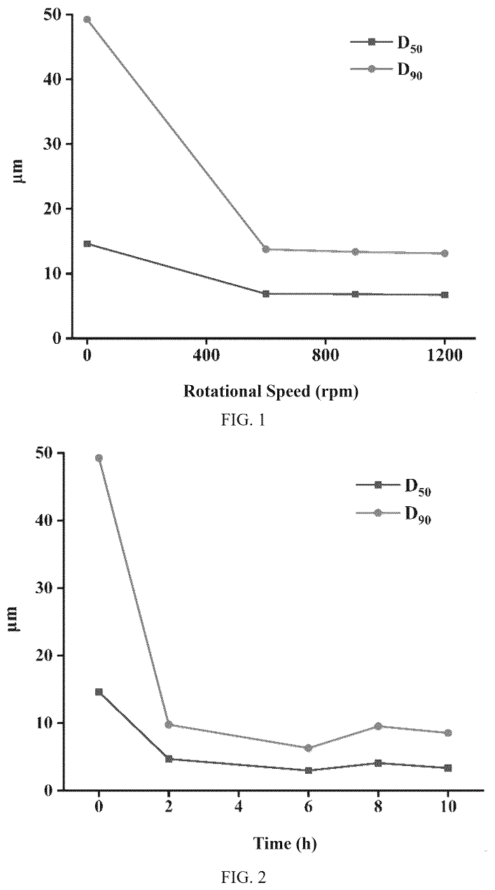

FIG. 1 shows the effect of stirring speed on the particle size of synephrine DPI. Compared with the API, increasing the stirring speed can reduce the particle size of synephrine powder. Under stirring speed conditions of 600-1200 rpm, the cumulative distribution of synephrine in the particle size range of 1-5 μm increased from 12.29% to 32.74%.

### 3.3 Effect of Stirring Time on Particle Size Distribution of Synephrine DPI

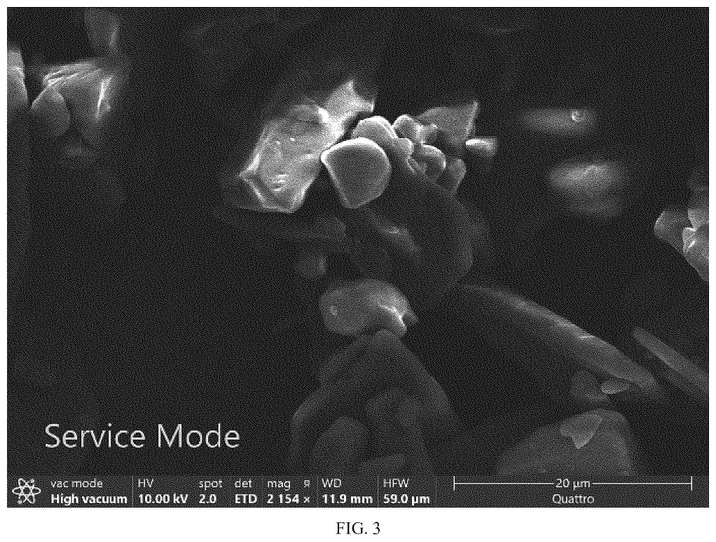

FIG. 2 shows the effect of stirring time on the particle size of synephrine DPI. Extending the stirring time can effectively reduce the particle size of synephrine DPI. When the stirring time is 6 h, the cumulative distribution in the particle size range of 1-5 μm reaches a maximum of 70.40%.

### 3.4 Particle Size Distribution

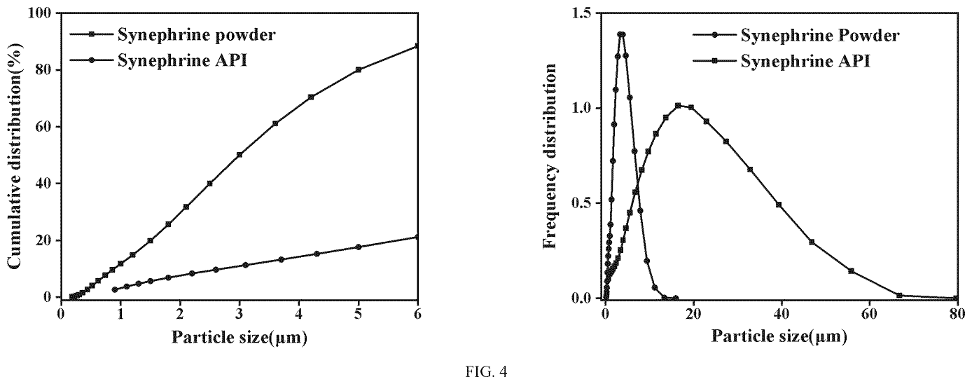

FIG. 4 shows the accumulation and frequency distribution of synephrine at 600 rpm for 6 h. It can be seen that after antisolvent precipitation, the distribution of synephrine DPI significantly increased within the particle size range of 1-5 μm, and the particle size at D50 was less than 5 μm, which is within the suitable inhalation particle size range, beneficial for effective lung deposition of the DPI.

### II. Characterization and Quality Evaluation of SYN-DPI

The SYN-DPI formulation was used as the target, scanning electron microscopy (SEM), PXRD, DSC, and FTIR were used to characterize SYN-DPI and evaluate the powder properties of the DPI to investigate whether it meets the requirements for DPIs. The in vitro deposition of the DPI was also examined.

### (1) Scanning Electron Microscopy (SEM)

Synephrine DPI was uniformly adhered to a conductive adhesive tape, sputtered with gold, and placed under an SEM. The sample was observed under conditions of a vacuum of 5×10−4 Pa, an electron beam working voltage of 10 kV, a working distance of approximately 10 mm, and a magnification of 1000-10000.

The morphology of SYN-DPI was observed using an SEM, as shown in FIG. 3. The SYN-DPI appeared as a sheet or block, with uniform size.

### (2) Powder X-Ray Diffraction (PXRD) Investigation

Appropriate amounts of synephrine API and DPI were weighed, respectively, ground into uniform powder, and prepared into flat sample specimens. The flat sample specimens were examined under the conditions of a copper target, a high voltage of 40 kV, a tube current of 100 mA, a scanning range of 10°-80° (20), and a scanning speed of 0.2°/min. PXRD patterns were plotted.

FIG. 5 shows the RXRD results of synephrine API and DPI. As can be seen from FIG. 5, both synephrine API and DPI have crystalline structures. Synephrine API exhibits strong diffraction peaks at 12.22°, 15.13°, 17.35°, 18.13°, 20.27°, 23.59°, 26.53°, 27.23°, 28.42°, and 31.47°. Synephrine DPI exhibits strong diffraction peaks at 12.12°, 15.12°, 17.35°, 18.09°, 20.25°, 23.61°, 26.47°, 27.21°, 28.40°, and 31.51°. Compared with synephrine API, the characteristic diffraction peak positions of DPI did not change, indicating that the crystal structure of synephrine did not change after antisolvent precipitation.

### (3) Differential Scanning Calorimetry (DSC) Investigation

5.41 mg of synephrine API and 2.23 mg of DPI were accurately weighed into an aluminum sample dish. The aluminum crucible was sealed using a stamping die and placed into the sample cell, along with a reference crucible. The purge atmosphere was set to high-purity nitrogen at a flow rate of 40 mL/min to maintain a dry N₂ environment. After the system stabilized, the initial temperature was set to 20° C. and programmed temperature rise detection was performed within the range of 20-350° C. at a temperature rise rate of 20.0° C./min. The DSC curves for the samples were plotted.

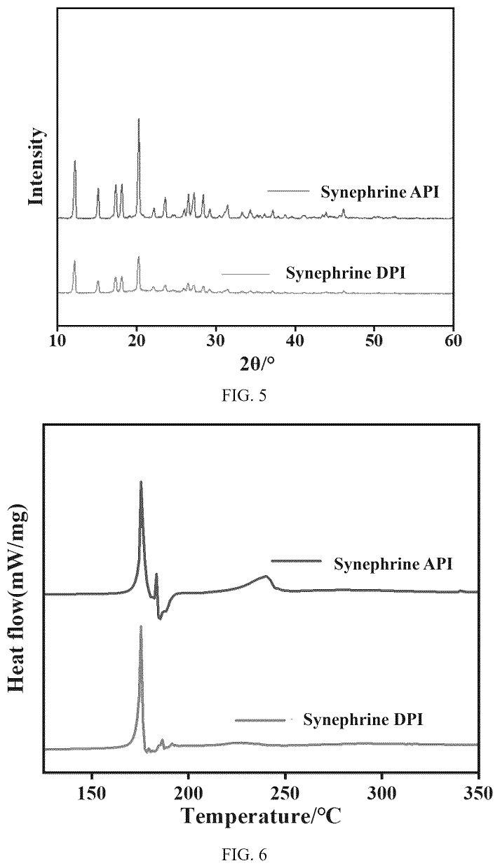

FIG. 6 shows the DSC results of synephrine API and DPI. Synephrine API exhibits an exothermic peak at 175° C., a small exothermic peak at 179° C., and a broad exothermic peak at 233-242° C. Synephrine DPI shows an exothermic peak at 175.44° C. and a small exothermic peak at 186° C., indicating that the crystal form of synephrine DPI remains unchanged after antisolvent precipitation. The disappearance of the broad endothermic melting peak at 233-242° C. suggests that the antisolvent precipitation method can not only prepare DPIs but also has a purification effect.

### (4) Fourier Transform Infrared Spectroscopy (FTIR) Investigation

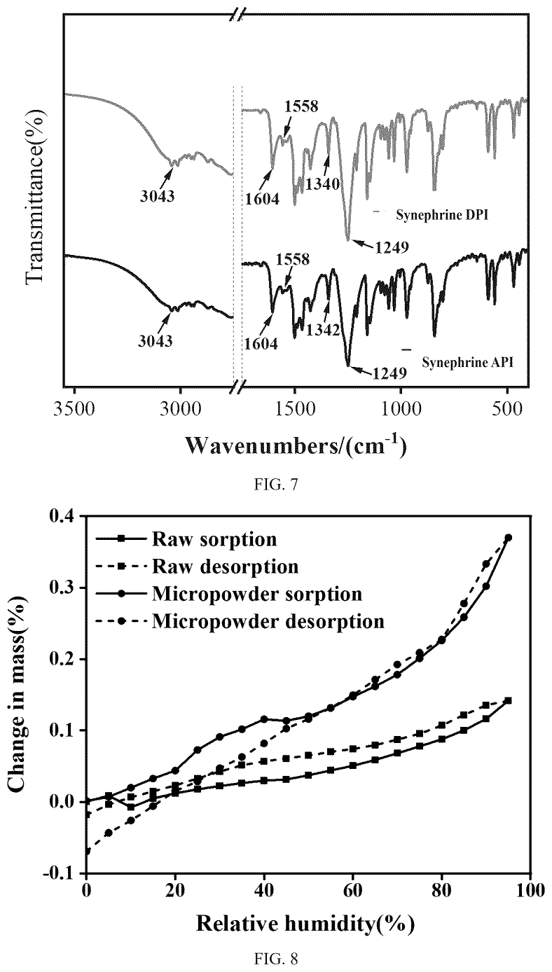

Approximately 2.00 mg of synephrine API and DPI and 200 mg of KBr powder were weighed, respectively, and ground in a mortar. The powder was placed in a tablet press mold, the mold was assembled, and the tablet was pressed. The resulting transparent tablet was then placed in a sample transmission measurement frame and scanned in the wavelength range of 3500-500 cm⁻¹, with a resolution of 0.09 cm⁻¹ and 16 scans, using potassium bromide as the background. The FTIR of synephrine API and DPI is shown in FIG. 7. As seen in FIG. 7, synephrine API and DPI have characteristic absorptions at 3043 cm⁻¹ (ν C—H), 1604 cm⁻¹ (ζ C—H), 1558 cm⁻¹ (ζ C═C), 1340 cm⁻¹ (ζ O—H, N—H), and 1249 cm⁻¹ (ν C═O), indicating that both synephrine API and DPI micropowders are crystals and their crystal form did not change after antisolvent precipitation.

### (5) Hygroscopicity Investigation

The hygroscopicity of synephrine API and DPI was analyzed using a dynamic vapor sorption (DVS) analyzer. The experimental temperature was set to room temperature (25° C.), the carrier gas was nitrogen, and the total gas flow rate was 200 mL/min. Samples were weighed and placed on the balance tray of the DVS analyzer. The relative humidity (RH) change program was set as required, and the humidity equilibrium time was also set.

The moisture adsorption curves of synephrine API and DPI are shown in FIG. 8. The results showed that the maximum moisture adsorption of the synephrine API under 90% RH conditions was 0.12%. According to the “9103 Guidelines for Hygroscopicity Testing of Drugs” in Part IV of the 2020 edition of the Chinese Pharmacopoeia, it belongs to samples with no or almost no hygroscopicity (hygroscopic weight gain less than 0.2%). The synephrine DPI showed a moisture absorption weight gain of 0.30% under 90% RH conditions, classifying it as a slightly hygroscopic sample (hygroscopic weight gain less than 2% but not less than 0.2%). This may be due to the small particle size of the obtained micropowder, resulting in a large surface area in contact with the external environment and rapid moisture diffusion, thus leading to strong hygroscopicity. Meanwhile, under normal storage conditions (60% RH) for both the synephrine API and DPI, the moisture absorption weight gain percentage was less than 0.2%, indicating that they are essentially non-hygroscopic. This meets the requirements for the production, transportation, and storage of DPIs, making them suitable for pulmonary drug delivery systems. During delivery, they will not cause particle aggregation or deposition when encountering moisture in the respiratory system.

### (6) Determination of Effective In Vitro Site Deposition Rate

The in vitro pulmonary deposition of synephrine DPI samples was investigated using the next-generation pharmaceutical impactor (NGI). The various components of the instrument were connected sequentially, including the NGI device, an adapter, an artificial throat, a pre-separator, and a collection tray. After installation, the airtightness and uniformity of the device were checked. The gas flow rate was 75 L/min. At this gas flow rate, the cutoff values Da of each stage of the collection tray were as follows: S1=7.145 μm, S2=3.971 μm, S3=2.522 μm, S4=1.495 μm, S5=0.835 μm, S6=0.481 μm, S7=0.293 μm, and the micro-orifice collector (MOC)=0.104 μm.

The specific method was as follows: 10 mg synephrine micropowder and API were added to No. 3 hydroxypropyl methylcellulose capsules, respectively, and then loaded into a single-capsule inhalation device. After connecting to an NGI, the measurement was performed. Five capsules were inhaled each time. The inhalation flow rate was 75 L/min, and the inhalation duration was 3.2 s. Drug powder deposited at each stage of the NGI was collected using ultrapure water. The synephrine content was determined under UV conditions, and the FPF and respirable fraction (RF) were calculated.

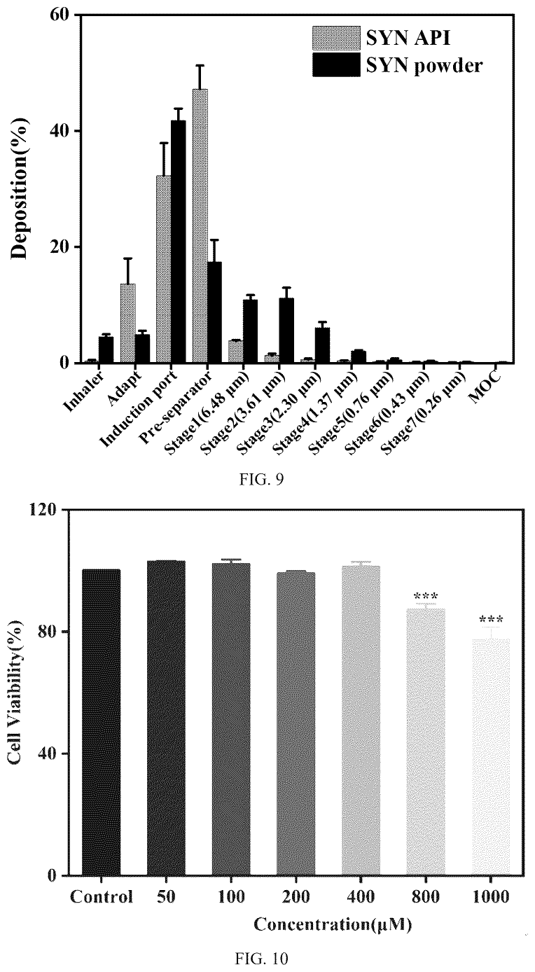

The deposition of synephrine API and DPI at each stage of the NGI is shown in FIG. 9. The results indicate that after antisolvent precipitation, the emptying rates of synephrine API and DPI were 90.98% and 95.51%, respectively. The FPFs were 2.69% and 31.39%, respectively. While the RFs were 41.26% and 65.41%, respectively. The effective in vitro lung deposition rates were 2.25% and 19.27%, respectively, meeting the requirements for dry powder inhalation.

### Example 2: In Vitro and In Vitro Pharmacodynamic Study of Synephrine Dry Powder Inhaler (SYN-DPI) on ALI

First, the cytotoxicity of SYN-DPI was investigated in vitro. A cellular inflammation model was constructed using lipopolysaccharide (LPS), and the in vitro efficacy of SYN-DPI was preliminarily evaluated using the enzyme-linked immunosorbent assay (ELISA). Then, an ALI model was established in rats using the tracheal instillation LPS method. The therapeutic effect of SYN-DPI on ALI was evaluated by detecting indicators such as pulmonary edema in rats, observing lung tissue pathological sections, and measuring the expression of oxidative factors and inflammatory cytokines in lung tissue homogenates and serum. Immunohistochemistry of lung tissue was combined to analyze the relevant protein pathways of SYN-DPI treatment of ALI.

### 1 Experimental Cells and Animals

Mouse mononuclear macrophages (RAW264.7 cells) were used in this experiment. Specific pathogen free (SPF)-grade male Sprague-Dawley (SD) rats (weighing 200-220 g) were used as experimental animals and were acclimatized for 7 days. Water was allowed for drinking 12 hours before the experiment, but fasting was required.

### 2 Experimental Methods

### 2.1 Cell Culture

### 2.1.1 Cryopreservation of RAW264.7 Cells

Cells in the good logarithmic growth phase were selected. The old culture medium was removed and the cell cryopreservation solution was prepared. The cell concentration was adjusted to 5×10⁶ cells/mL. The cells were aliquoted into cryovials, the cryovials were placed in a programmed cooling box. After 24 hours, the cryovials were removed and stored in a liquid nitrogen tank.

### 2.1.2 Thawing of RAW264.7 Cells

The constant temperature water bath was heated to 37° C. The cryopreserved cells were quickly placed into the water bath and shaken continuously for quick thawing. After thawing, the cells were transferred to centrifuge tubes and mixed with at least 10 times the volume of fresh culture medium. The mixture was centrifuged at 1250 rpm for 5 mins, and the supernatant was discarded. The Dulbecco's modified eagle medium (DMEM) culture medium containing 10% fetal bovine serum (FBS) was used to resuspend the cells, and the resuspended cells were seeded into culture dishes.

### 2.1.3 Culture of RAW264.7 Cells

Cells were cultured in DMEM culture medium containing 10% FBS at 37° C. in a 5% CO₂ incubator.

### 2.2 In Vitro Anti-Inflammatory Assay Using SYN-DPI

### 2.2.1 Cytotoxicity Assay

The inhibitory effect of different concentrations of SYN-DPI on the survival of RAW264.7 cells was studied using the cell counting kit (CCK)-8 assay. A logarithmically growing RAW264.7 cell suspension was seeded into 96-well plates, 100 μL per well, and cultured for 24 hours in a cell culture incubator at 37° C. with 5% CO₂. Fresh culture medium was added to the blank control group, while different concentrations of the test drug were added to the experimental groups. Six replicates were set up for each group, 100 μL per well, and the plates were incubated in the cell culture incubator for another 24 hours. The remaining liquid was aspirated, and 10 μL of CCK-8 reagent was added to each well. The plates were then incubated in the culture incubator for another 1-2 hours, and the absorbance was measured at 450 nm using a microplate reader.

$$cell viability ( \% ) = \frac{{A}_{sample} - {A}_{blank}}{{A}_{control} - {A}_{blank}} \times 100 \%$$

### 2.2.2 Establishment of the Cellular Inflammation Model

RAW264.7 cells in logarithmic growth phase were seeded at 100 μL per well in a 96-well plate and cultured at 37° C. and 5% CO₂ for 24 hours. The supernatant was aspirated and discarded. 100 μL of medium containing synephrine at an appropriate concentration was added to each well for pretreatment for 1 h, followed by the addition of LPS-containing medium. The cells were then co-incubated with the test substance for 24 hours.

### 2.2.3 Determination of Cellular Inflammatory Cytokines

Based on the CCK-8 assay results, RAW264.7 cells in logarithmic growth phase were seeded in 96-well plates and divided into the control group, LPS group, LPS+SYN group, and LPS+SYN-DPI group. A cellular inflammation model was established. After culturing for 24 h, the supernatant from each well was collected and centrifuged at 3500 rpm for 10 min. The supernatant was collected and the subsequent procedures were strictly performed according to the ELISA kit instructions. The absorbance was measured at 450 nm using a microplate reader, and the contents of interleukin (IL)-1B, IL-6, tumor necrosis factor (TNF)-α, and nitric oxide (NO) were calculated.

### 2.3 Experimental Methods for Improving LPS-Induced ALI Model in Rats Using SYN-DPI

### 2.3.1 Construction of LPS-Induced ALI Model in Rats

Male SD rats were acclimatized for one week, and an ALI model in rats was established using tracheal instillation of LPS. The specific modeling steps were as follows: the rats were first anesthetized with isoflurane, then the rats were fixed in a supine position to facilitate intratracheal administration to the lungs. LPS (8 mg/kg) was nebulized into the alveoli using a microsprayer. The microsprayer was then removed, the animal was placed in a vertical position, and gently rotated for 30 seconds to ensure even distribution of LPS throughout the lungs.

### 2.3.2 Grouping and Administration of Rats with ALI

Referring to relevant literature on synephrine, the maximum human dose of synephrine is 100 mg. Based on the clinically equivalent dose, the dose administered to the rats via gavage was 9 mg/kg, and the low-dose inhalation group was 4.5 mg/kg. The administration details for each group are shown in Table 3.

TABLE 3

| | Administration | Administration | Administration | Route of |
|---|---|---|---|---|
| Group | Concentration(mg/kg) | Dosage(mg) | Volume (mL) | Administration |
| Blank Control | 0.9% Normal | / | 2 | Gavage |
| Group | Saline | | | |
| Model Group | 0.9% Normal | / | 2 | Gavage |
| Positive | Saline | | | |
| Control Drug | 3 | 0.6 | 2 | Gavage |
| Dex Group | | | | |
| API Group | 9 | 1.8 | 2 | Gavage |
| SYN-DPI Low | 4.5 | 0.9 | / | Inhalation |
| Dose Group | | | | |
| SYN-DPI | 9 | 1.8 | / | Inhalation |
| Medium | | | | |
| Dose Group | | | | |
| SYN-DPIHigh | 18 | 3.6 | / | Inhalation |
| Dose Group | | | | |

### 2.3.3 Peripheral Blood Collection

Six hours after drug administration, rats were anesthetized by intraperitoneal injection of sodium pentobarbital. Blood was collected from the abdominal aorta in sterile blood collection tubes, gently shaken, and allowed to stand. The tubes were then centrifuged at 3500 rpm for 15 mins, and the serum was separated and stored in a −80° C. freezer for the detection of serum inflammatory cytokine levels.

### 2.3.4 Bronchoalveolar Lavage Fluid Collection

After blood collection, the rats were placed in a supine position on the operating table until their hearts stopped beating. The skin of the chest and abdomen, sternum, and surrounding soft tissues were sequentially cut with tissue scissors to expose the lung tissue below the glottis, including the main bronchus. The right lung was ligated, and a small inverted V-shaped incision was made in the trachea with surgical scissors. A syringe was carefully inserted through the incision for lavage. The left lung was instilled with physiological saline three times via the trachea, 1.5 mL each time, and aspirated after each slow instillation. The bronchoalveolar lavage fluid was collected.

### 2.3.5 Lung Tissue Collection

After instillation of the left lung of the rats, the left and right lungs were collected, washed with physiological saline, and dried. The lower lobe of the left lung was used to determine the wet-to-dry weight ratio of the rat lung tissue. The upper lobe of the right lung was temporarily stored in liquid nitrogen and transferred to an −80° C. freezer for oxidative factor detection. The remaining right lung was stored in 4% paraformaldehyde for histopathological examination and immunohistochemical analysis.

### 2.3.6 Lung Tissue Wet-to-Dry Weight Ratio Measurement

The lower lobe of the left lung of the rat was collected, surface blood was removed, surface moisture was wiped off, and the lobe was weighed. The wet weight was recorded. The lobe was then dried in a 60° C. oven for 24 hours, and the dry weight was recorded. The degree of pulmonary edema was evaluated by calculating the lung wet/dry weight ratio.

Lung wet/dry weight ratio=Left lung wet weight/Left lung dry weight.

### 2.3.7 Hematoxylin-Eosin (HE) Staining

Lung tissue fixed in 4% paraformaldehyde was dehydrated, routinely embedded in paraffin, and prepared into 5 μm sections. After dewaxing, the sections were routinely stained with HE, dehydrated, mounted, and observed under a microscope to examine the structure and morphological characteristics of the rat lung tissue in each group.

### 2.3.8 Masson Staining

The sections were routinely dewaxed to water. Hydrophobic zones were drawn, and Bouin's solution was added dropwise. The sections were then covered and incubated at 37° C. After incubation, they were rinsed with running water until the yellow color disappeared. Next, the sections were stained with celestine blue staining solution for 3-5 mins, followed by rinsing with running water. After completion, the sections were stained with Mayer's hematoxylin staining solution for 3 min, followed by rinsing with running water. After completion, differentiation was performed using acidic ethanol differentiation solution for a few seconds, followed by rinsing with running water. Then the sections were stained with ponceau-fuchsin staining solution for 3 min, followed by a brief rinse with distilled water. Finally, the sections were treated with phosphotungstic acid solution for 5-10 min, the supernatant was poured off, and then the sections were stained with aniline blue staining solution in decreasing increments for 5 min. Afterward, the sections were treated briefly with a weak acid solution. Upon completion, the routine dehydration was performed, and the sections were cleared with xylene. Finally, mounting was carried out with neutral resin to complete Masson's trichrome staining.

### 2.3.9 Determination of Protein Content in Bronchoalveolar Lavage Fluid (BALF)

This study used the bicinchoninic acid (BCA) assay to determine the total protein content in BALF. The tissue weight was accurately measured, and physiological saline was added according to the weight-to-volume ratio. After homogenization and centrifugation of the lung tissue, the supernatant was collected. The test solution was added sequentially according to the protein quantification kit instructions. The absorbance was measured at 562 nm using a microplate reader, and the protein concentration in BALF was calculated.

### 2.3.10 Detection of Serum Inflammatory Cytokine Levels

Rat serum was collected, and the levels of TNF-α, IL-6, IL-8, IL-1β, and IL-1α were measured separately according to the rat ELISA kit instructions. The OD value was measured at 450 nm using a microplate reader. A standard curve was plotted with the concentrations of TNF-α, IL-8, IL-6, IL-1β, and IL-1α standards on the x-axis and the absorbance values on the y-axis. The concentrations of TNF-α, IL-6, IL-8, IL-1β, and IL-1α in serum were calculated based on the standard curve.

### 2.3.11 Detection of Oxidative Stress Indicators in Lung Tissue

A certain weight of rat lung tissue was accurately weighed. Samples for detecting activities of superoxide dismutase (SOD), catalase (CAT), glutathione (GSH), and malondialdehyde (MDA) were prepared according to different ratios of lung tissue weight to physiological saline. The mixture was homogenized under ice-water bath conditions and then centrifuged. The supernatant was collected for detection after the centrifugation. The kit instructions were strictly followed.

### 2.3.12 Immunohistochemical Analysis of Lung Tissue

Lung tissue sections were dewaxed until transparent, rinsed with ultrapure water, and set aside. Subsequently, the sections were placed in 0.01 M sodium citrate buffer solution for 15 mins to perform antigen extraction. Following this, the sections were incubated in 3% hydrogen peroxide for 10 mins to block endogenous peroxidase activity. After blocking, the sections were incubated overnight at 4° C. with the primary antibodies Nrf2, NF-κB, and p38 MAPK. After primary antibody incubation, the sections were blotted dry, and a secondary antibody was applied to cover the lung tissue on the sections, followed by incubation at room temperature for 1 h. The sections were washed with phosphate buffered saline (PBS), blotted to dry, and the prepared chromogenic reagent was added. The chromogenic reaction was terminated by rinsing the sections with ultrapure water. Hematoxylin staining was performed for 5 min to counterstain the cell nuclei, followed by a rinse with ultrapure water. After differentiation with hematoxylin differentiation solution for 10 s, the sections were rinsed again. Hematoxylin bluing solution was applied for bluing, followed by a rinse with ultrapure water. Finally, the sections were dehydrated with anhydrous ethanol and xylene until transparent, mounted with neutral resin, and observed under a microscope for image acquisition.

### 3. Experimental Results

### 3.1 Cytotoxicity

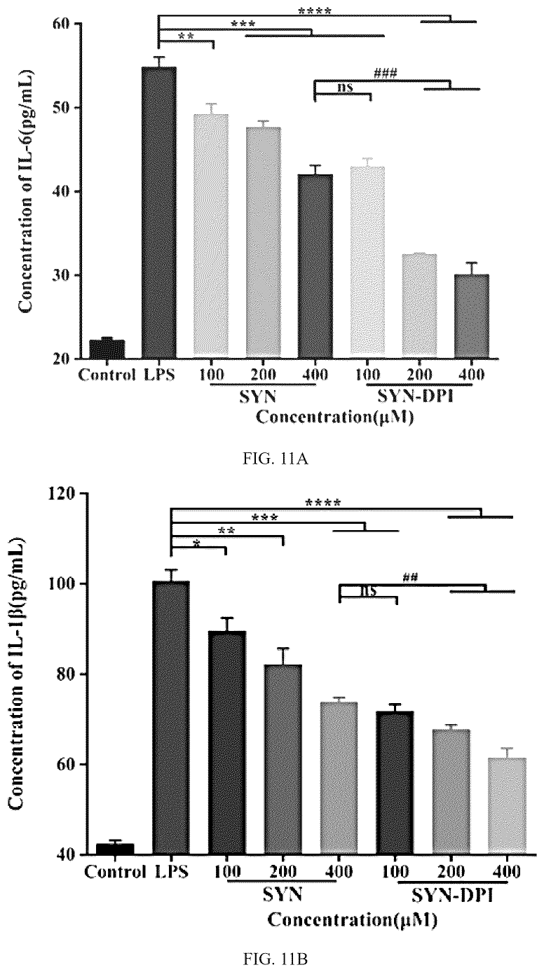

The cytotoxicity results of SYN-DPI on RAW264.7 cells are shown in FIG. 10. The results showed that SYN-DPI did not cause any significant cytotoxicity in the concentration range of 50-400 μM and had almost no effect on the viability of RAW264.7 cells. However, at concentrations of 800 μM and 1000 μM, cell viability decreased (87.3±1.6% and 77.8±3.8% respectively compared to the control group), exerting adverse effects on RAW264.7 cell viability, with a significant difference (P<0.001). Therefore, we selected a concentration range of 50-400 μM for subsequent experiments.

### 3.2 Effect of SYN-DPI on Expression of Pro-Inflammatory Cytokines in an LPS-Induced In Vitro Inflammation Model

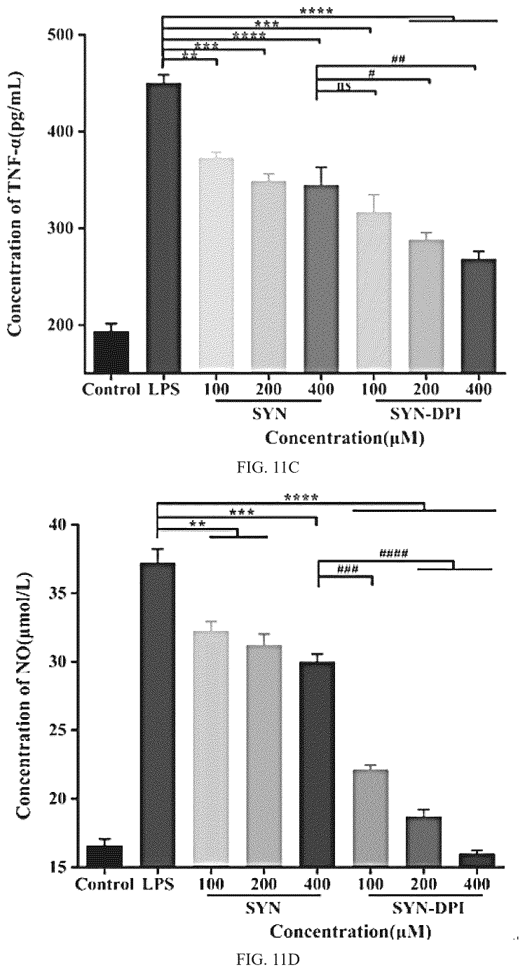

The effect of SYN-DPI on the expression of pro-inflammatory cytokines in an LPS-induced cellular inflammation model is shown in FIGS. 11A-11D. The results showed that, compared with the control group, RAW264.7 cells stimulated with LPS produced substantial amounts of IL-1β, IL-6, and TNF-α with significant differences, indicating a successful establishment of the cellular inflammation model. Taking IL-6 as an example, there was no significant difference between the 100 μM SYN-DPI group and the 400 μM SYN group (P>0.05), indicating that SYN-DPI at a dose of 100 μM can achieve a similar inhibitory effect on inflammatory cytokine expression as 400 μM SYN. The inhibition rate of IL-6 production by SYN-DPI (400 μM) was 45.17%, while the inhibition rate of IL-6 production by the same amount of SYN (400 μM) was 23.32%, suggesting that SYN-DPI has better anti-inflammatory potential. Nitric oxide (NO) is a reactive free radical involved in the inflammatory process. SYN (200 μM) inhibited NO production by 16.08%, while SYN-DPI (200 μM) inhibited it by 49.77%, showing a higher inhibitory effect than SYN. In short, both SYN and SYN-DPI inhibited NO production, but SYN-DPI showed a better inhibitory effect. Therefore, within the scope of this study, SYN-DPI had a better anti-inflammatory effect compared to SYN.

### 3.3 Effect of SYN-DPI on Wet-to-Dry Weight Ratio of Rat Lungs

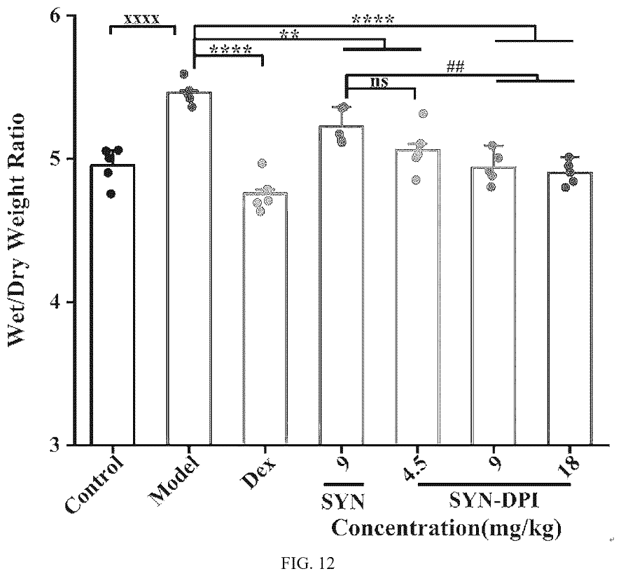

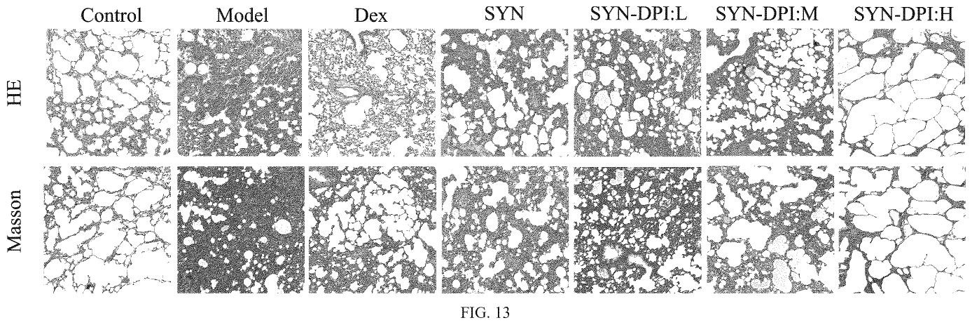

The results of the wet-to-dry weight ratio of rat lungs are shown in FIG. 12. As seen in FIG. 12, the wet/dry weight ratio of the lungs in the model group was significantly higher than that in the control group and the drug-treated group, indicating a significant difference, and suggesting that the LPS-induced ALI rat model was successfully established. Compared to the model group, both the SYN and SYN-DPI groups showed a significant reduction in the wet-to-dry weight ratio of rat lungs, with the ability of the SYN-DPI group to improve pulmonary edema in rats being dose-dependent. The low-dose SYN-DPI group showed no significant difference in its ability to reduce pulmonary edema compared to the SYN group (P>0.05), indicating that the low-dose SYN-DPI achieved the same efficacy as SYN. Furthermore, the ability of SYN-DPI to reduce pulmonary edema was stronger than that of SYN.

### 3.4 Effect of SYN-DPI on the Pathological Changes of Lung Tissue in LPS-Induced Rat ALI Model

The pathological changes observed by HE and Masson staining are shown in FIG. 13. In the Control group, the bronchial ciliated columnar epithelial structure of the lung tissue was clear, and the alveolar cavity structure was intact. In the Model group, the alveolar cavities of the rats were significantly reduced, showing extremely severe protein and inflammatory cell infiltration, alveolar wall thickening, alveolar edema, and even congestion and degeneration. The pathological damage of the rat lung tissue in the Dex group was significantly alleviated. There was no significant difference in pathological changes between the SYN group and the low-dose SYN-DPI group. Compared with the Model group rats, the low-dose SYN-DPI group rats showed a reduction in inflammatory cells in the lung tissue, the medium-dose group rats showed smooth bronchial epithelium with some inflammatory cell infiltration around the bronchi, and the high-dose group rats showed a normal alveolar cavity with largely normal inflammatory cells. This indicates that SYN-DPI can alleviate LPS-induced lung tissue pathological damage in a dose-dependent manner.

### 3.5 Effect of SYN-DPI on Total Protein Content in BALF of ALI Rats

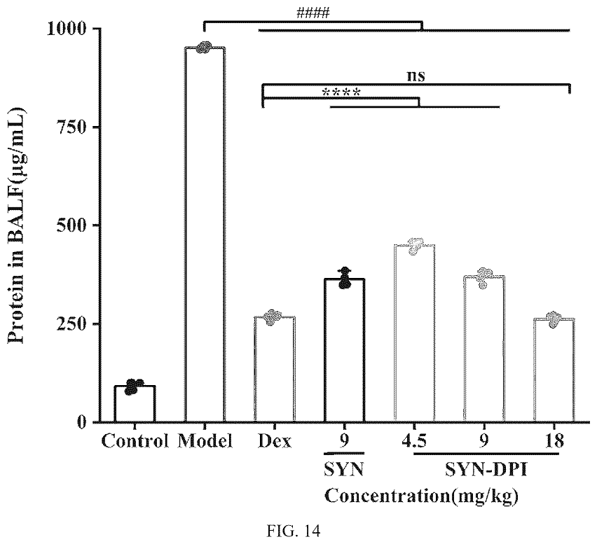

The effects of SYN and SYN-DPI on the total protein content in BALF of ALI rats are shown in FIG. 14. As shown in FIG. 14, compared with the blank control group, the protein content in the BALF of the model group significantly increased, indicating a significant difference and an increase in alveolar interstitial space, thus successfully establishing the ALI model. The protein content in the drug-treated groups was significantly lower than that in the LPS group, indicating that SYN-DPI and the positive control drug Dex have similar therapeutic effects and can significantly improve alveolar interstitial space. Data analysis comparing the low, medium, and high-dose SYN-DPI groups revealed that the protein content in BALF was closely related to the drug dosage and showed a dose-dependent effect.

### 3.6 Effects of SYN-DPI on Serum Inflammatory Cytokines in LPS-Induced ALI in Rats

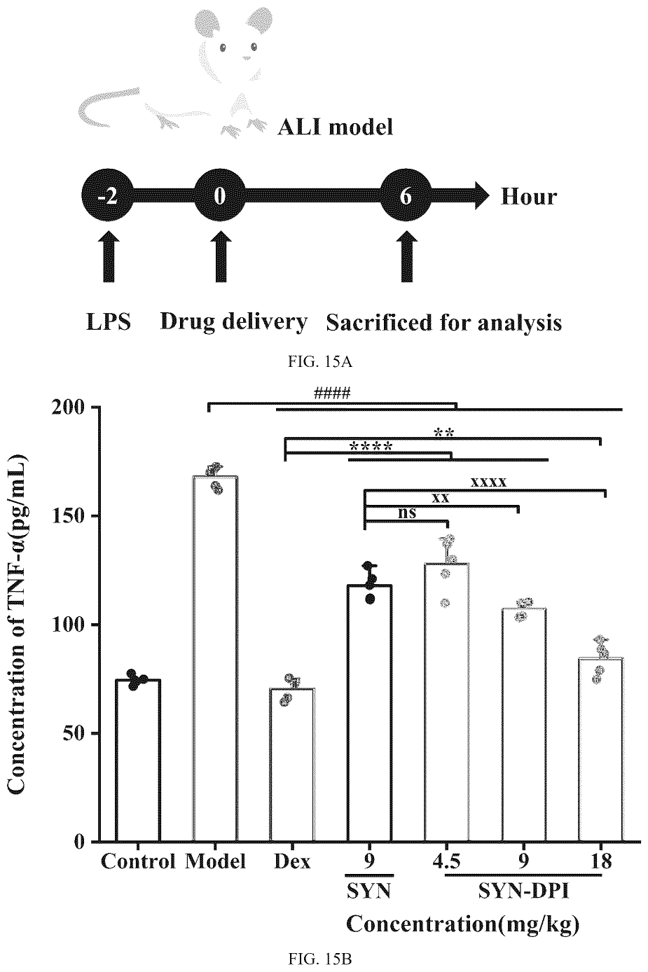

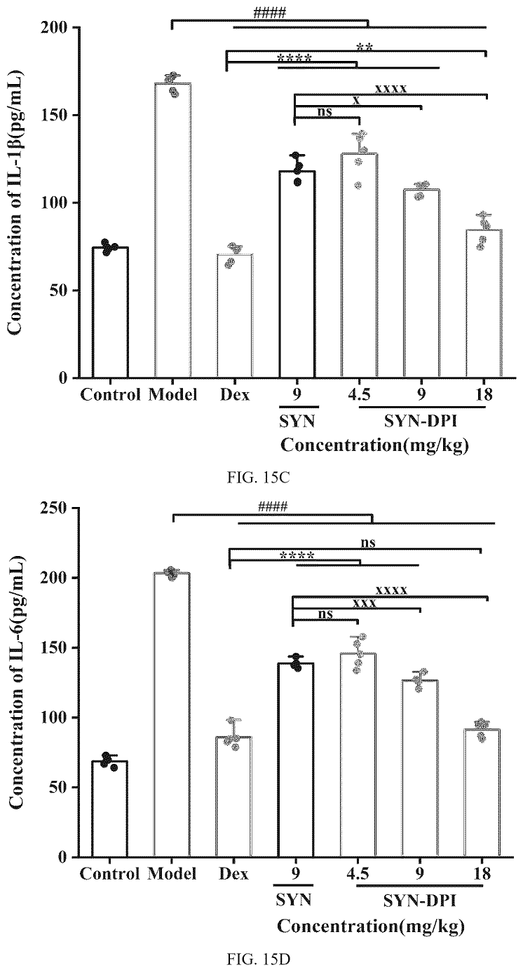

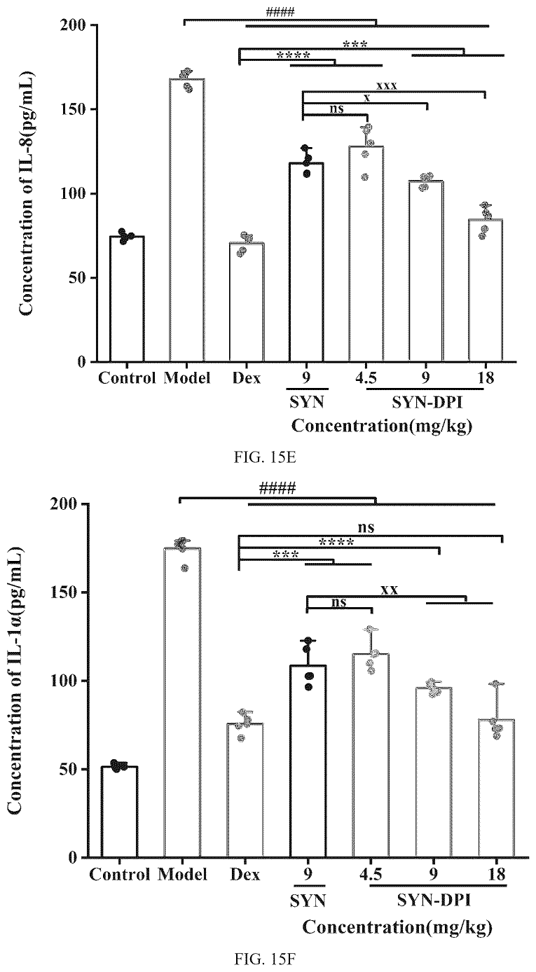

The results of serum IL-1a, IL-1β, IL-6, IL-8, and TNF-α levels are shown in FIGS. 15A-15F. Compared to the blank control group, the secretion of inflammatory cytokines in the LPS-induced model group significantly increased (P<0.0001). After treatment with SYN and SYN-DPI, the expression levels of inflammatory cytokines in the SYN group, SYN-DPI group, and Dex group were significantly decreased compared to the model group (P<0.0001), indicating that both SYN and SYN-DPI can improve ALI and reduce the expression levels of inflammatory cytokines in the serum, exhibiting significant therapeutic effects. Taking TNF-α as an example, there was no significant difference between the low-dose SYN-DPI group (4.5 mg/kg) and the SYN group (9 mg/kg) (P>0.05), indicating that the low-dose SYN-DPI group could achieve a similar inhibitory effect on the expression of inflammatory cytokines as the SYN group. Analysis of the SYN-DPI group data also revealed that the secretion of inflammatory cytokines was dose-dependent; increasing the dosage within the experimental concentration range could dose-dependently reduce the secretion of inflammatory cytokines, thus inhibiting LPS-induced inflammatory responses in rats.

### 3.7 Effects of SYN-DPI on Oxidative Factors in LPS-Induced ALI Lung Tissue of Rats

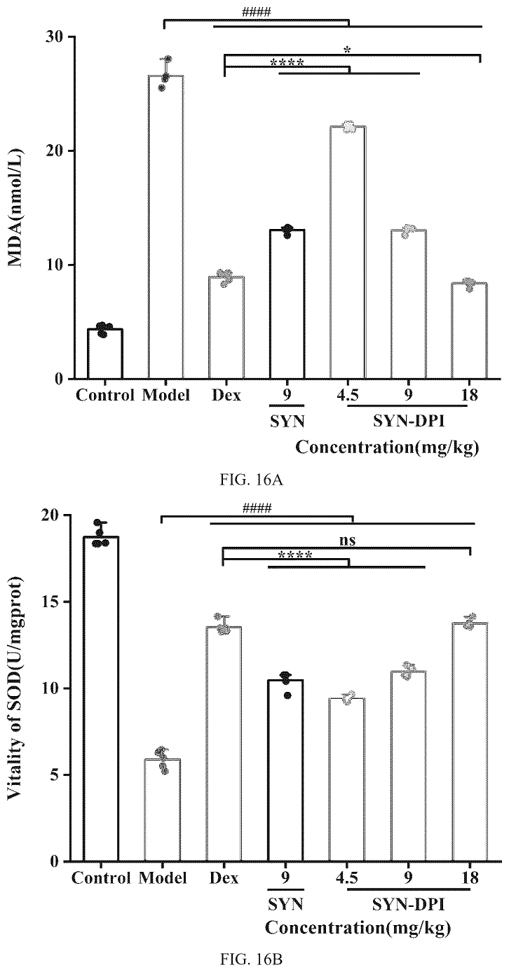

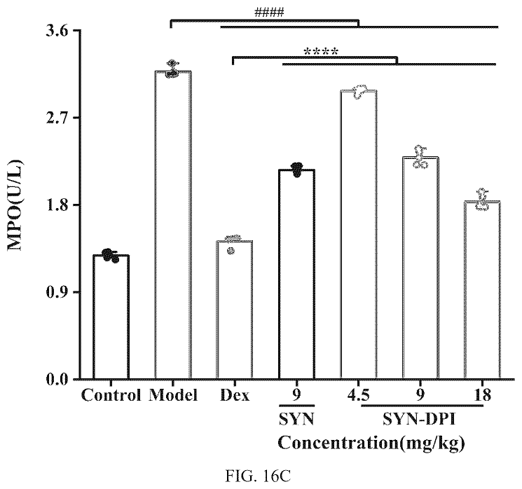

The effects of SYN and SYN-DPI on MDA, SOD, and myeloperoxidase (MPO) in LPS-induced ALI lung tissue of rats are shown in FIGS. 16A-16C. The results showed that compared with the blank control group, LPS stimulation in the model group significantly increased MDA and MPO levels and decreased SOD activity. SYN-DPI treatment could significantly reverse the effects of LPS on redox-related enzymes, reduce peroxidation metabolites, enhance antioxidant levels, and maintain oxidative stress balance.

### 3.8 Effects of SYN-DPI on the Expression of NF-κB, Nrf2, and p38 MAPK Proteins in ALI Rat Lung Tissue

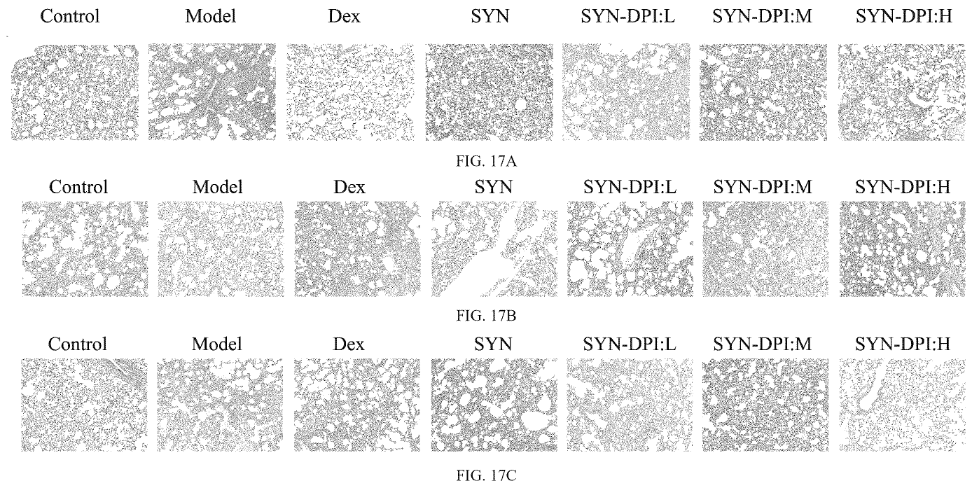

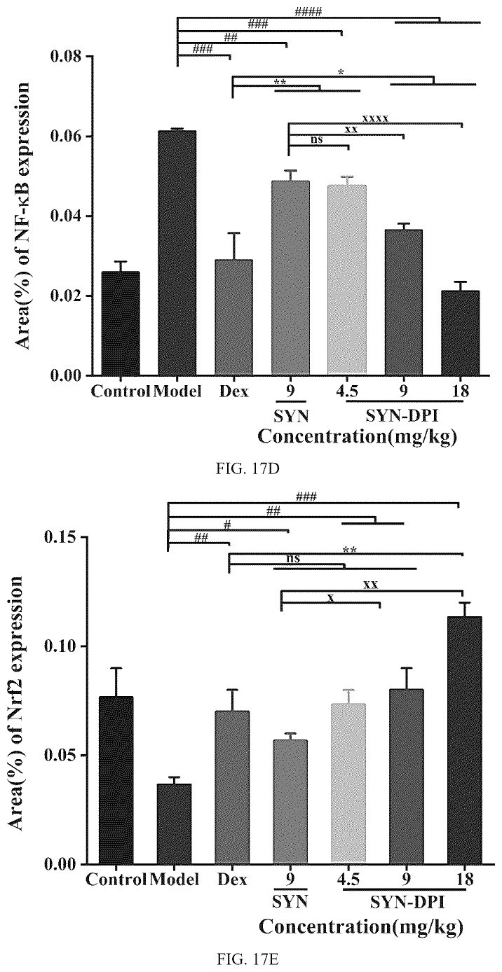

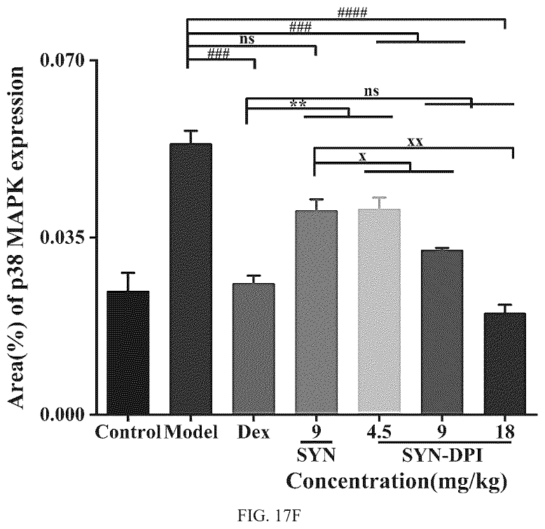

Immunohistochemical analysis and microscopic imaging revealed the expression of NF-κB, Nrf2, and p38 MAPK proteins, as shown in FIGS. 17A-17F. ImageJ software was used for semi-quantitative analysis of the expression of NF-κB, Nrf2, and p38 MAPK proteins, with mean optical density as the metric. NF-κB and MAPK are two classic inflammatory signaling pathways; activation of the NF-κB and MAPK pathways promotes the production of pro-inflammatory mediators. Therefore, inhibiting the NF-κB and MAPK pathways helps suppress the production of pro-inflammatory cytokines. In an LPS-induced ALI model, the effects of SYN-DPI on the NF-κB and MAPK signaling pathways were further investigated. The results showed that LPS significantly increased the expression of p38 MAPK protein, while synephrine dry powder inhalation significantly inhibited protein phosphorylation, indicating that synephrine exerts its anti-inflammatory effect through the MAPK signaling pathway. As a classic antioxidant pathway, LPS can block the Nrf2 signaling pathway and inhibit the expression of downstream antioxidant genes. As shown in FIGS. 17A-17F, compared with the blank control group and the LPS-treated group, SYN-DPI treatment effectively promoted Nrf2 nuclear transcription, indicating that SYN-DPI can effectively activate Nrf2.

### Example 3: Pharmacokinetic Study of Synephrine DPI

To investigate the changes in SYN-DPI concentration after absorption into the bloodstream, this chapter proposes to establish a simple, sensitive, and rapid liquid chromatography-tandem mass spectrometry (LC-MS/MS) method to determine the concentration of the compound SYN in rat plasma. The method will be rigorously validated based on quantitative analysis of drugs in biological samples to explore the metabolic process of SYN in rats, providing a foundation for further research on SYN-DPI.

### 1 Experimental Animals

SPF-grade male SD rats, weighing 180-220 g, were housed under identical experimental conditions: temperature (22±1)° C., relative humidity (55±5) %, and a 12-hour light/dark cycle. They had free access to food and water and underwent a one-week acclimatization period before the experiment.

### 2 Experimental Methods

### 2.1 Chromatographic Conditions

Column: Hypersil GOLD C8 (100 mm×2.1 mm; 3 μm); Mobile phase: Phase A: 0.1% formic acid aqueous solution, Phase B: acetonitrile; Flow rate: 0.3 mL/min; Column temperature: 40° C.; Injection volume: 2 μL; Gradient elution program: 0-1 min, 5% B; 1-4 min, 5%-90% B; 4-5 min, 90%-5% B.

### 2.2 Mass Spectrometry Conditions

Mass spectrometry was performed in electrospray ionization (ESI) positive ion scanning mode, using multiple reaction monitoring (MRM) detection mode. The collision gas was high-purity nitrogen; capillary voltage: 3.5 kV; nebulizer pressure: 45 psi; desiccator temperature: 300° C.; drying gas flow rate: 5 L/min (N₂); sheath gas temperature: 250° C.; sheath gas flow rate: 11 L/min (N₂).

### 2.3 Experimental Animal Grouping and Administration

Experimental animals were randomly divided into three groups: (1) tail vein injection group, (2) pulmonary inhalation group, and (3) gavage group, with 5 animals in each group. Animals were fasted for 12 h before administration but had free access to water.

### 2.4 Plasma Sample Collection

Rats were fasted for 12 hours before the experiment but were allowed free access to water. The rats were weighed to calculate the dosage and numbered. After administration, 0.2 mL of blood was collected from the orbital vein at time points of 2 min, 5 min, 10 min, 15 min, 30 min, 1 h, 2 h, 3 h, 4 h, and 6 h. The blood samples were placed in an EP tube containing heparin sodium and were centrifuged at 3500 rpm for 15 min. The supernatant plasma was separated and stored at −80° C. freezer.

### 2.5 Plasma Sample Detection

190 μL of a rat plasma sample was accurately pipetted into an EP tube. Then, 10 μL of an internal standard solution was added, and the sample was vortexed for 60 s. Next, 400 μL of acetonitrile was added, and the sample was vortexed for 60 s to precipitate proteins. The sample was then centrifuged at 10000 rpm for 15 mins, and the supernatant was passed through a 0.22 μm microporous filter membrane. Finally, the filtered sample was placed in a glass insert for LC-MS/MS analysis.

### 3 Experimental Results

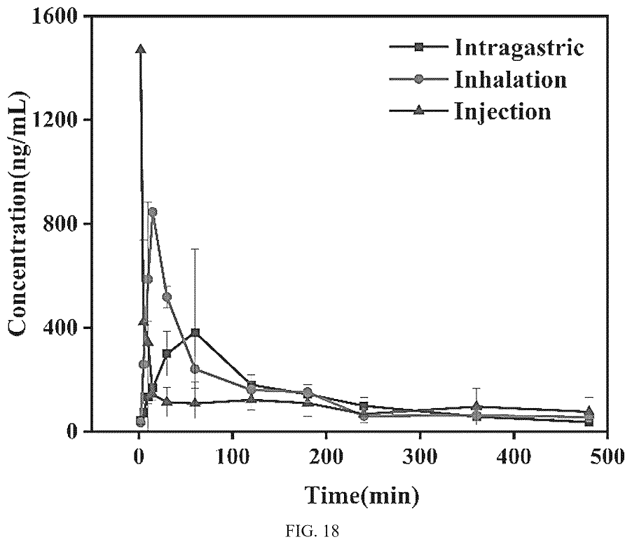

The synephrine-dose curve is shown in FIG. 18. The results showed that, compared with the gavage group, after administering the same dose of synephrine DPI via pulmonary administration, the Cₘₐₓ of the inhalation group was 845.52 ng/ml, while that of the gavage group was 301.76 ng/ml, with the inhalation group being 280.20% higher than the gavage group; the half-life t½ of the inhalation group was 175.51 min, while that of the gavage group was 137.65 min, with the inhalation group being 127.50% longer than the gavage group; the area-under-the-curve (AUC)₍₀₋ₜ₎ of the inhalation group was 1168.24 ng/ml*h, while that of the gavage group was 876.07 ng/ml*h, with the inhalation group being 133.35% higher than the gavage group. Compared with the injection group, after administering the same dose of synephrine DPI via pulmonary administration, the Cₘₐₓ of the injection group was 1032.17 ng/mL, the inhalation group reached 81.92% of the injection group, and the gavage group reached 29.24% of the injection group; the half-life (t½) of the injection group was 320 min, the inhalation group reached 54.84% of the injection group, and the gavage group reached 43.01% of the injection group; the AUC_{(0-%)} of the inhalation group was 1335.70 ng/ml*h, the AUC_{(0-∞)} of the injection group was 1538.76 ng/ml*h, the inhalation group reached 86.80% of the injection group, and the gavage group was only 64.48% of the injection group. The absolute bioavailability of the gavage group was 32.24%, and the absolute bioavailability of the inhalation group was 86.80%. Therefore, based on the pharmacokinetic parameters of synephrine in different administration groups, inhalation administration can significantly improve its bioavailability.

What is claimed is:

1. An application of a synephrine dry powder inhaler in preparation of a medicament for treating acute lung injury; wherein
a method for preparing the synephrine dry powder inhaler comprises the following steps:
(1) dissolving a synephrine active pharmaceutical ingredient in a good solvent to forma good solvent phase A, and using n-hexane as an antisolvent phase B;
(2) adding the good solvent phase A dropwise to the antisolvent phase B under stirring conditions to obtain a uniform suspension; and
(3) centrifuging the suspension obtained in the step (2), and subsequently precipitating and drying to obtain the synephrine dry powder inhaler;
wherein the good solvent is anhydrous ethanol; and a stirring speed is 600-1200 rpm, and a stirring time is 2-10 h.

2. The application according to claim 1, wherein an aerodynamic particle size of the synephrine dry powder inhaler is 1-5 μm.

3. The application according to claim 1, wherein the synephrine dry powder inhaler has a fine particle fraction of 28-33%, an emptying rate of 94-98%, an inhalable fraction of 60-70%, and an effective in vitro lung deposition rate of 18-20%.

4. The application according to claim 1, wherein in the step (1), a concentration of the good solvent phase A is 10-20 mg/mL.

5. The application according to claim 1, wherein in the step (2), a volume ratio of the good solvent phase A to the antisolvent phase B is 1:8-12.

6. The application according to claim 1, wherein in the step (3), a drying method is constant temperature drying at 30-40° C.
